(19) United States
(12) Patent Application Publication
(10) Pub. No.: US 2010/0121350 A1
(43) Pub. Date: May 13, 2010

(54) INSTANTANEOUS MECHANICAL DETACHMENT MECHANISM FOR VASO-OCCLUSIVE DEVICES

(76) Inventor: Greg Mirigian, Dublin, CA (US)

Correspondence Address: VISTA IP LAW GROUP LLP, 12930 Saratoga Avenue, Suite D-2, Saratoga, CA, 95070, US

(21) Appl. No.: 12/450,681

(22) PCT Filed: Mar. 12, 2008
(86) PCT No.: PCT/US08/03234

§ 371 Date: Oct. 6, 2009

Related U.S. Application Data
(60) Provisional application No. 60/923,141, filed on Apr. 12, 2007.

(51) Int. Cl. A61B 17/10; A61B 17/08

(52) U.S. Cl. 606/142; 606/158

(57) ABSTRACT

Disclosed herein are mechanical detachment mechanisms for vaso-occlusive devices that allow for instantaneous, operator-controlled release of the vaso-occlusive device into the selected site. Also disclosed are vaso-occlusive assemblies comprising these detachment mechanisms and methods of using these detachment mechanisms and vaso-occlusive assemblies.

### TECHNICAL FIELD

Compositions and methods for repair of aneurysms are described. In particular, mechanical detachment mechanisms for instantaneous detachment of an embolic device and vaso-occlusive assemblies comprising these detachment mechanisms are described.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.). Variations of such devices include polymeric coatings or attached polymeric filaments have also been described. See, e.g., U.S. Pat. Nos. 5,226,911; 5,935,145; 6,033,423; 6,280,457; 6,287,318; and 6,299,627. In addition, coil designs including stretch-resistant members that run through the lumen of the helical vaso-occlusive coil have also been described. See, e.g., U.S. Pat. Nos. 5,582,619; 5,833,705; 5,853,418; 6,004,338; 6,013,084; 6,179,857; and 6,193,728.

Coils have typically been placed at the desired site within the vasculature using a catheter and a pusher. The site is first accessed by the catheter (e.g., small diameter catheters such as those shown in U.S. Pat. Nos. 4,739,768 and 4,813,934). The catheter may be guided to the site through the use of guidewires (see U.S. Pat. No. 4,884,579) or by flow-directed means such as balloons placed at the distal end of the catheter.

Once the site has been reached, the catheter lumen is cleared by removing the guidewire (if a guidewire has been used), and one or more coils are placed into the proximal open end of the catheter and advanced through the catheter with a pusher. Once the coil reaches the distal end of the catheter, it is discharged from the catheter by the pusher into the vascular site. However, there are concerns when discharging the coil from the distal end of the catheter. For example, the plunging action of the pusher and the coil can make it difficult to position the coil at the site in a controlled manner and with a fine degree of accuracy. Inaccurate placement of the coil can be problematic because once the coil has left the catheter, it is difficult to reposition or retrieve the coil.

Several techniques involving Interlocking Detachable Coils (IDCs), which incorporate mechanical release mechanisms and Guglielmi Detachable Coils (GDCs), which utilize electrolytically actuated release mechanisms, have been developed to enable more accurate placement of coils within a vessel.

IDCs, for example as described in U.S. Pat. No. 5,261,916, include mating structures on the pusher and coil that interlock when constrained by the catheter or a coaxial sleeve structure. When the restraining coaxial member is moved away from the junction of the interlocking parts, the coil is freed from the catheter assembly and the pusher may then be removed.

Another DC device for placement of coils is shown in U.S. Pat. No. 5,234,437. This device includes a coil having a helical portion at least one end and a pusher wire having a distal end that is threaded inside of the helical coil by use of a threaded section on the outside of the pusher. The device operates by engaging the proximal end of the coil with a sleeve and unthreading the pusher from the coil. Once the pusher is free, the sleeve may be used to push the coil out into the targeted treatment area.

Electrolytic coil detachment is disclosed in U.S. Pat. Nos. 5,122,136; 5,354,295; 6,620,152; 6,425,893; and 5,976,131, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint. The coil is bonded via a metal-to-metal joint to the distal end of the pusher. The pusher and coil are made of dissimilar metals. The coil-carrying pusher is advanced through the catheter to the site and a small electrical current is passed through the pusher-coil assembly. The current causes the joint between the pusher and the coil to be severed via electrolysis. The pusher may then be retracted leaving the detached coil at an exact position within the vessel. Since no significant mechanical force is applied to the coil during electrolytic detachment, highly accurate coil placement is readily achieved. In addition, the electric current may facilitate thrombus formation at the coil site. The disadvantage of this method is that the electrolytic release of the coil may require a period of time that may inhibit rapid detachment of the coil from the pusher.

Another method of placing an embolic coil is by thermally detachable mechanism. U.S. Pat. No. 5,578,074 describes a thermally activated shape memory decoupling mechanism. U.S. Pat. No. 5,108,407 shows the use of a device in which embolic coils are separated from the distal end of a catheter by the use of heat-releasable adhesive bonds. The coil adheres to the therapeutic device via a mounting connection having a heat sensitive adhesive. Laser energy is transferred through a fiber optic cable which terminates at that connector. The connector becomes warm and releases the adhesive bond between the connector and the coil. Among the drawbacks of this system is that it involves generally complicated laser optic componentry.

There is a need to provide alternative mechanisms for delivering implants, such as embolic coils, that combine accurate positioning capability with rapid implant decoupling response times.

### SUMMARY

Disclosed herein are instantaneously detachable embolic devices, as well as methods of using and making these devices. The vaso-occlusive devices are detachably linked to a pusher mechanism via a detachment mechanism. In the first engaged position, the detachment mechanism engages the vaso-occlusive device (which may be modified at or near the proximal end to engage the detachment mechanism), even when it is extruded from the delivery device (e.g., catheter). When the vaso-occlusive device is in the desired position in the site to occluded, the operator (surgeon) can change the detachment mechanism to a second position such that the detachment mechanism no longer engages the vaso-occlusive device and the device is instantaneously released into the desired site. The pusher and detachment mechanism can then be withdrawn, leaving the vaso-occlusive device in the desired position.

Thus, unlike previously-described interlocking detachment mechanisms, the detachment mechanisms described herein do not release the vaso-occlusive device when extruded from a coaxial restraining member, but, instead requires operator action to switch the mechanism from the first engaged position to the second unengaged position.

In one aspect, provided herein is a detachment mechanism adapted to detachably engage a vaso-occlusive device, the detachment mechanism comprising at least one arm, the arm having first and second positions, wherein in the first position, the arm engages the vaso-occlusive device and, in the second position, the arm releases the vaso-occlusive device. In certain embodiments, the detachment mechanism further comprises an actuator that moves the arm between the first and second positions.

Any of the detachment mechanisms described herein may have two or more arms, for example, 2, 3, 4, 5, 6 or even more arms. In certain embodiments, one or more of the arms are curved. Furthermore, in any of the detachment mechanisms described herein a mandrel can be used to engage the arm with the vaso-occlusive device, for example, by separating the arms of a detachment mechanism comprising two or more arms.

In any of the detachment mechanisms described herein, one or more of the arms may further comprise a ball-like structure at its distal end. The arms and/or optional ball-like structure(s) may be made of one or more metals and/or polymers.

In another aspect, provided herein is a detachment mechanism adapted to detachably engage a vaso-occlusive device, the detachment mechanism comprising at least one arm having first and second positions, wherein in the first position, the arm engages the vaso-occlusive device and, in the second position, the arm releases the vaso-occlusive device, and a means for moving the detachment mechanism between the first and second positions.

In yet another aspect, provided herein is vaso-occlusive assembly comprising: a vaso-occlusive device having proximal and distal ends; a pusher element having proximal and distal ends; any of the detachment mechanisms described herein at the distal end of the pusher element, wherein the arm(s) of the detachment mechanism has(have) a distal end and proximal end. In certain embodiments, the distal end of the arm engages the vaso-occlusive device and the proximal end of the arm is embedded in the pusher element. Furthermore, in certain embodiments, the vaso-occlusive assemblies as described herein further comprise a removeable mandrel that engages the arm of the detachment mechanism with the vaso-occlusive device.

In any of the assemblies described herein, the vaso-occlusive device may comprise a helically wound vaso-occlusive coil (e.g., metal and/or polymer). In these embodiments, the arm(s) may engage(s) one or more proximal windings of the helically wound coil, for example by modifying the pitch of one or more proximal windings of the coil to engage the arm.

In certain embodiments, the vaso-occlusive device engages an anchor structure (e.g., ring or loop) at the proximal end of the vaso-occlusive device. The anchor structure may extend into the vaso-occlusive device.

In yet another aspect, provided herein is a method of at least partially occluding an aneurysm, the method comprising the steps of introducing any of the vaso-occlusive assemblies described herein into the aneurysm, wherein the detachment mechanism is in the engaged position; and switching the detaching mechanism to the unengaged position, thereby instantaneously deploying the vaso-occlusive device into the aneurysm.

These and other embodiments will readily occur to those of skill in the art in light of the disclosure herein.

### BRIEF DESCRIPTION OF THE FIGURES

FIG. 1 is a partial cross-section, side view depicting an exemplary vaso-occlusive assembly as described herein. The detachment mechanism is shown in the position in which it engages the vaso-occlusive device to be delivered.

FIG. 2 is a partial cross-section, side view depicting the exemplary vaso-occlusive assembly of FIG. 1 and showing the detachment mechanism in the position in which it does not engage the vaso-occlusive device.

FIG. 3, panels A and B, are front views of exemplary assemblies as described herein having 2 detachment arms terminating in spherical ball-like structures. FIG. 3A shows the arms separated by a mandrel and the spherical ball-like structures engaging the vaso-occlusive coil. FIG. 3B shows the 2-armed device after withdrawal of the mandrel. The arms of the detachment mechanism no longer engage the vaso-occlusive coil.

FIG. 4, panels A and B, are front views of exemplary assemblies as described herein having 3 detachment arms terminating in spherical ball-like structures. FIG. 4A shows the arms separated by a mandrel and the spherical ball-like structures engaging the vaso-occlusive coil. FIG. 4B shows the 3-armed device after withdrawal of the mandrel when the detachment mechanism no longer engages the vaso-occlusive coil.

FIG. 5, panels A and B, are front views of exemplary assemblies as described herein having 4 detachment arms terminating in spherical ball-like structures. FIG. 5A shows the arms separated by a mandrel and the spherical ball-like structures engaging the vaso-occlusive coil. FIG. 5B shows the 4-armed device after withdrawal of the mandrel when the detachment mechanism no longer engages the vaso-occlusive coil.

FIG. 6 is a partial cross-section, side view depicting an exemplary assembly in which an additional element adapted to engage a detachment mechanism as described herein is secured to the proximal end of a vaso-occlusive coil. Also shown on the attachment is a ring-like (eyelet) structure extending into the lumen of the coil through which a filament can be threaded for enhancing stretch-resistance of the vaso-occlusive coil.

FIG. 7, panels A and B, are overviews of exemplary actuating mechanisms that allow the operator to switch the detachment mechanism from a first (engaged) position to a second (unengaged) position.

### DETAILED DESCRIPTION

Instantaneous detachment mechanisms for occlusive (e.g., embolic) devices and assemblies are described. The detachment mechanisms described herein can be utilized in devices useful in vascular and neurovascular indications and are particularly useful in delivering such embolic devices to aneurysms, for example small-diameter, curved or otherwise difficult to access vasculature, for example aneurysms, such as cerebral aneurysms. Methods of making and using these detachments and assemblies comprising these detachments are also aspects of this disclosure.

Currently, the gold-standard method of delivering vaso-occlusive devices is via electrolytic detachment (e.g., GDC coils). While electrolytic detachment solves the drawbacks of earlier mechanical detachments (e.g., the need for the mechanism to be fully inside the catheter in order to remain engaged), electrolytically detachable coils typically require approximately 20-30 seconds detachment times.

Unlike previously-described mechanically detachable assemblies, the vaso-occlusive assemblies described herein do not unintentionally detach when extended from the catheter. Furthermore, unlike electrolytically detachable assemblies, the devices described herein detach instantaneously when actuated by the operator (surgeon).

Therefore, advantages of the present disclosure include, but are not limited to, (i) the provision of instantaneously detachable vaso-occlusive devices; (ii) the provision of mechanically detachable implantable devices that can be extended beyond the catheter tip, thereby allowing for more precise placement of the devices; and (iii) the provision of occlusive devices that minimize the mechanical motion needed to detach the devices.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to a device comprising “an arm” includes devices comprising of two or more such arms.

The vaso-occlusive assemblies described herein comprise a vaso-occlusive device detachably connected to a pusher via a mechanically detachable mechanism.

The structure of the detachment mechanism is preferably such that it can instantaneously release the vaso-occlusive device from the pusher. Typically, the detachment mechanism is permanently connected to the pusher, for example by bonding the detachment mechanism to the distal end of the pusher or by creating a pusher element with an integral detachment mechanism at this distal end. The detachment mechanism may be attached to anywhere to the pusher, as long as it does not interfere with delivery and/or withdrawal of the pusher.

The detachment mechanism may comprise metal (e.g., nitinol, stainless steel) and/or polymeric materials. In certain embodiment, the detachment mechanism comprises a super-elastic metal alloy such as nitinol which allows for durability and flexibility. Stainless steel or other metals or alloys can also be used. A portion or all of the detachment mechanism may include one or more surface treatments (coating, machining, microtexturing, etc.).

The detachment mechanism has at least two positions. In the first position, the detachment mechanism engages the vaso-occlusive device and allows the pusher-detachment mechanism and vaso-occlusive device to be moved as a unit, even when the vaso-occlusive device is extruded from the end of the delivery mechanism (e.g., delivery catheter). In the second position, the detachment mechanism does not engage the vaso-occlusive device, which is immediately released into the selected site.

The detachment mechanism may be designed so that the default position of the detachment mechanism is the unengaged position. For example, as shown in the Figures, the detachment mechanism may comprise arms that are structured to engage the vaso-occlusive device. The arms are then engaged with the vaso-occlusive device, for example by placing a mandrel or other structural element between the arms and/or by forcing the arms apart with an actuator. To release the vaso-occlusive device, the operator switches the detachment junction to the default unengaged (closed arm) position, for example, by removing the structural element holding the arms open and/or releasing pressure on the actuator and allowing the detachment mechanism to return to the default (unengaged) position.

Alternatively, the detachment mechanism may be configured to have a default engaged position. For example, the exemplary arm structure shown in the Figures could be designed such that the “relaxed” position is open and engaged with the vaso-occlusive device. In these embodiments, an actuator would be configured so that the operator closes the arms to release the vaso-occlusive device. The detachment mechanism may be retained in the unengaged position during withdrawal of the pusher.

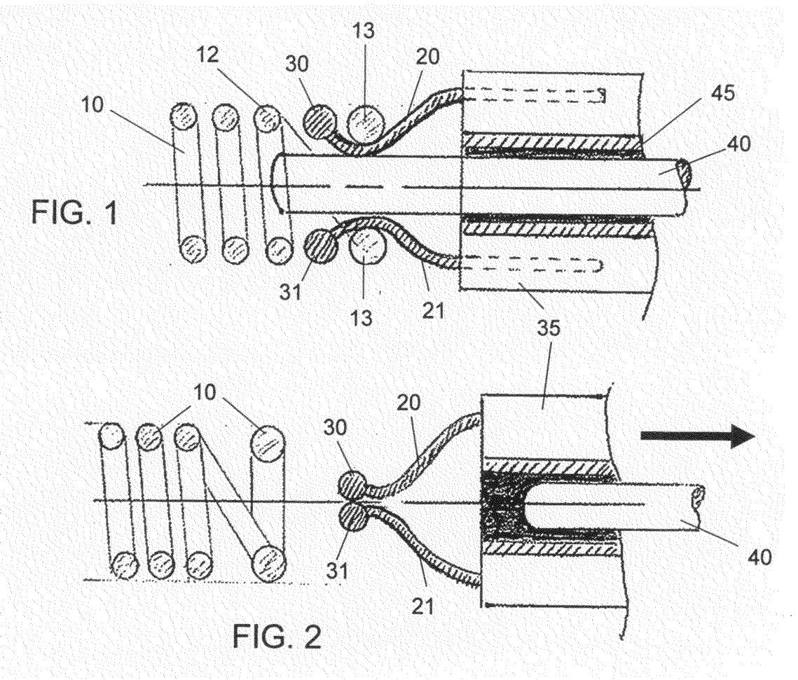

The detachment mechanism may take any desired shape. In certain embodiments, the detachment mechanism comprises arms, one or more of which may comprise a double curved shape (see, e.g., FIG. 1), which allows the arms to curl around the proximal end of the embolic device. As noted above, the arms may be made of any metal (e.g., nitinol) and/or polymer and the proximal ends of the arm may be permanently or detachably connected to the pusher element, for example embedded into the distal walls of the pusher and/or secured with adhesive.

The detachment mechanism may include any number of “arms.” A skilled artisan can readily determine how many arms should be used by taking into account factors such as the size of the vaso-occlusive device, the nature of the site to be occluded, etc. For example, for −18 type coils (typically having an outer diameter of 0.015″ and an inner diameter of about 0.011″), two arms with spherical ball distal elements of ˜0.004-0.005″ may be used to slide through the proximal coil end in the closed position upon pusher retraction. Alternatively, for the same coil, detachment mechanisms with three or four arms with balls of smaller diameters may also be used. Designs with more than one arm (e.g., 2, 3, 4, 5, 6 or even more arms) may allow for greater rotational movement and/or flexibility of the vaso-occlusive device with respect to the pusher-detachment mechanism while maintaining a secure connection therebetween.

The distal ends of the arms may include any structure to aid in engagement of the vaso-occlusive device, including but not limited to one or more spherical shapes, curved or hooked shapes, or the like. The optional distal end shape may be made of the same material as the rest of the arts or a different material. For example, in certain embodiments, the optional distal element is made of a different radioopaque material than the rest of the arms, which would allow the operator to visually distinguish between the first and second positions (e.g., the arms comprise nitinol and the distal element of the arms comprises platinum).

The optional distal element may be on one, some, or all of the arm(s) and may be integral to the arms or attached after construction of the arms. For example, a spherical ball-shape (with or without an aperture therethrough sized to fit the sphere over the arm) can be secured to the distal end of the arm by any means (e.g., adhesive, soldering, weldings, or other method).

The detachment mechanisms described herein also allow for ready retrieval and/or repositioning of vaso-occlusive devices.

As noted above, the detachment mechanism extends from the distal end of a pusher body. The pusher may be of any shape (e.g., tubular, cylindrical, etc.). Optionally, the pusher may include a lumen therethrough to allow for the insertion and retraction of an element which holds the detachment mechanism in the first or second position (e.g., a mandrel which, when inserted through the pusher, holds the detachment mechanism in a position such that it engages the vaso-occlusive device).

The pusher element may also be designed such that flexibility varies over the length (e.g., the distal portion being made more flexible than the proximal portion). Methods of varying flexibility are known to the skilled artisan and include varying the composition over the length of the pusher (e.g., polymer composite ratios), and/or linking two or more separate segments of varying flexibility.

The pusher element may optionally contain hypotube component that is distally flexible, for example a metallic hypotube comprising micromachined slots and/or a continuous spiral cuts. Such designs are available from Boston Scientific under the trade names Synchro™ microfabricated nitinol guidewires and Wingspan™ stent system. Methods of making these flexible components are described in the art, for example, in U.S. Pat. Nos. 7,122,048 and 7,052,492.

The pusher element may also comprises additional elements, including but not limited to, a jacket or liner (e.g., polymer), a metal reinforced polymer structure, one or more components that better secure the detachment mechanism, and/or one or more radioopaque elements (e.g., marker band(s)).

The optional additional structural element(s) that can be used to hold the detachment mechanism in the first or second position (e.g., a mandrel) may be made of a polymer and/or metal. In certain embodiments, the mandrel comprises a polymer (e.g., PTFE). The optional additional structural element is typically slightly longer than the pusher to allow for advancement into the proximal end of the vaso-occlusive device and for connection to a release/actuation mechanism at the proximal end of the pusher.

Furthermore, as noted above, switching between the first (engaged) and second (released) positions is controlled by the operator (surgeon) via an actuator connected to the detachment mechanism. Any actuator mechanism (button, sliding mechanism, lever, twisting mechanism, etc.) can be used and will be readily known to those of skill in the art. Additionally, actuators may include one or more handles, dials or the like with which the operator (e.g., surgeon placing the device) controls movement position of the detachment mechanism. The detachment mechanism may be attached, either directly or through another element such as a pusher wire, to an actuator.

The detachment mechanisms described herein can be adapted to be used with any vaso-occlusive devices, including, but not limited to, metal and/or polymeric devices. Suitable metals and metal alloys include the Platinum Group metals, especially platinum, rhodium, palladium, rhenium, as well as tungsten, gold, silver, tantalum, and alloys of these metals. The core element may also comprise of any of a wide variety of stainless steels. Very desirable materials of construction, from a mechanical point of view, are materials that maintain their shape despite being subjected to high stress including but not limited to “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.”

The detachment mechanisms described herein may be used with vaso-occlusive devices of any structure, for example, vaso-occlusive devices of tubular structures, for examples, braids, coils, combination braid and coils and the like. Thus, although depicted in the Figures described below as a coil, the vaso-occlusive device may be of a variety of shapes or configuration includes, but not limited to, braids, knits, woven structures, tubes (e.g., perforated or slotted tubes), cables, injection-molded devices and the like. See, e.g., U.S. Pat. No. 6,533,801 and International Patent Publication WO 02/096273. The vaso-occlusive device may change shape upon deployment, for example change from a constrained linear form to a relaxed, three-dimensional (secondary) configuration. See, also, U.S. Pat. No. 6,280,457. In a preferred embodiment, the core element comprises a metal wire wound into a primary helical shape.

The core element may be, but is not necessarily, subjected to a heating step to set the wire into the primary shape. Methods of making vaso-occlusive coils having a linear helical shape and/or a different three-dimensional (secondary) configuration are known in the art and described in detail in the documents cited above, for example in U.S. Pat. No. 6,280,457. Thus, it is further within the scope of this disclosure that the vaso-occlusive device as a whole or elements thereof comprise secondary shapes or structures that differ from the linear coil shapes depicted in the Figures, for examples, spheres, ellipses, spirals, ovoids, figure-8 shapes, etc. The devices described herein may be self-forming in that they assume the secondary configuration upon deployment into an aneurysm. Alternatively, the devices may assume their secondary configurations under certain conditions (e.g., change in temperature, application of energy, etc.).

FIG. 1 shows a partial cross-section, side-view of an exemplary mechanically detachable vaso-occlusive assembly as described herein in an engaged (open arm) position (e.g., vaso-occlusive device is engaged to pusher via detachment mechanism). In this position, the proximal region of vaso-occlusive coil 10 is adapted to engage the spherical ball structures 30, 31 on detachment element arms 20, 21 of detachment mechanism connected to the distal end of tubular pusher 35. Tubular pusher 35 comprises a lumen and a mandrel 40 extends through the lumen. The interior of the lumen optionally comprises a coating or liner that reduces friction 45. Mandrel 40 extends through the lumen of pusher 35 and between the arms 20, 21 of detachment mechanism so the that arms engage the coil 10 between the two proximal-most windings 12, 13 via the spherical ball like structures 30, 31.

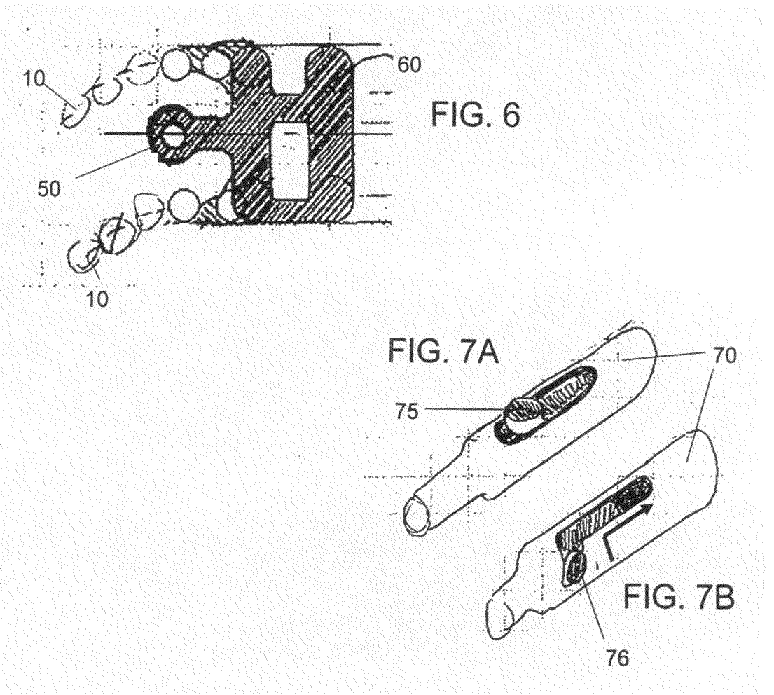

Although shown in FIG. 1 as engaged between the two most proximal coil windings, it will be apparent that the vaso-occlusive device may be adapted in any way to engage the detachment mechanism. Such modifications include changing the pitch of the coil windings to accommodate the detachment mechanism, and modifying the vaso-occlusive device to contain grooves, slots or other structures that are sized to fit the mechanical detachment junction in the first (engaged) position. See, also, FIG. 6.

FIG. 2 is a side and partial cross-section view of the vaso-occlusive assembly of FIG. 1 after release of the device by removal of the mandrel 40. Detachment mechanism arms 20, 21 are in a closed position without the mandrel 40 and, accordingly, no longer engage the windings of the coil 10, thereby allowing the pusher-detachment mechanism to be removed. The arrow shows the direction the mandrel is moved to release the vaso-occlusive device.

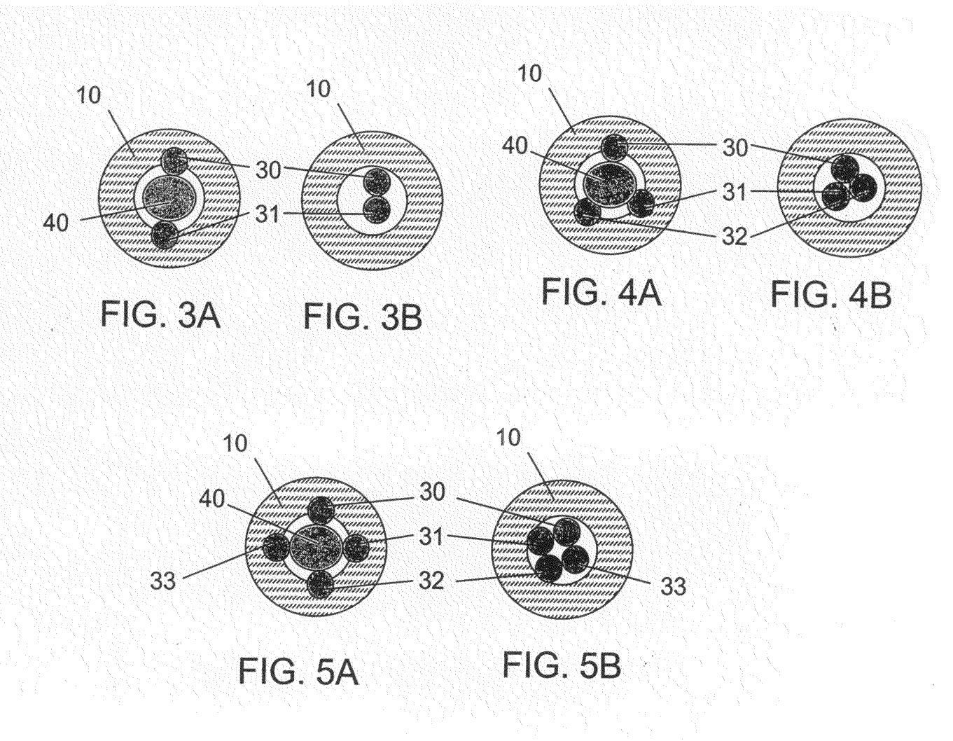

FIG. 3A is a front view of vaso-occlusive assembly as shown in FIGS. 1 and 2 in the engaged position. Two spherical ball-like structures 30, 31 at the distal end of the detachment mechanism arms engage the vaso-occlusive coil 10 when a mandrel 40 is inserted between the arms.

FIG. 3B is a front view of the two-arm embodiment of FIG. 3A in the unengaged (closed) position when the mandrel is withdrawn. Ball-like structures 30, 31 no longer engage vaso-occlusive coil 10 and pusher-detachment mechanism can be removed.

FIG. 4A is a front view of an exemplary vaso-occlusive assembly having 3 detachment mechanism arms. Each arm has a spherical ball-like structure 30, 31, 32 at the distal end of the detachment mechanism arms which engage the vaso-occlusive coil 10 when a mandrel 40 is inserted between the arms, positioning the ball-like structures 30, 31, 32 so that they engage the coil 10.

FIG. 4B is a front view of the exemplary 3-armed detachment mechanism of FIG. 4A in the unengaged (closed) position when the mandrel is withdrawn. Ball-like structures 30, 31, 32 no longer engage vaso-occlusive coil 10 and pusher-detachment mechanism can be removed.

FIG. 5A is a front view of an exemplary vaso-occlusive assembly having 4 detachment mechanism arms. Each of the 4 arms has a spherical ball-like structure 30, 31, 32, 33 at the distal end of the detachment mechanism arms which engages the vaso-occlusive coil 10 when a mandrel 40.

FIG. 5B is a front view of the exemplary 4-armed detachment mechanism of FIG. 5A in the unengaged (closed) position when the mandrel is withdrawn. Ball-like structures 30, 31, 32, 33 no longer engage vaso-occlusive coil 10 and pusher-detachment mechanism can be removed.

FIG. 6 shows an exemplary modification that can be made to the proximal end of a coil 10. Grooved element 60 is secured to the proximal end of the coil and includes areas for engagement of a detachment mechanism. Also shown on the modified proximal end element 60 is a ring structure 50 for stretch-resistant devices as described, for example, in U.S. patent application Ser. No. 11/400,100, filed Apr. 5, 2006.

FIGS. 7A and B show exemplary actuating mechanism for switching the detachment mechanism between the first and second positions. FIG. 7A shows a button (or lever) that can be pulled back by the operator to release the vaso-occlusive devices. FIG. 7B shows another exemplary actuator in which the operator moves the mechanism in an L-shaped manner (bold arrow) to release the vaso-occlusive.

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the absorbable vaso-occlusive device at the distal end, is advanced through the catheter.

Once the selected site has been reached, the vaso-occlusive device is extruded using a pusher-detachment mechanism as described herein and released in the desired position of the selected site.

Modifications of the procedure and vaso-occlusive devices described above, and the methods of using them in keeping with this disclosure will be apparent to those having skill in this mechanical and surgical art. These variations are intended to be within the scope of the claims that follow.

1. A detachment mechanism adapted to detachably engage a vaso-occlusive device, the detachment mechanism comprising
at least one arm, the arm having first and second positions, wherein in the first position, the arm engages the vaso-occlusive device and, in the second position, the arm releases the vaso-occlusive device.

2. The detachment mechanism of claim 1, further comprising an actuator that moves the arm between the first and second positions.

3. The detachment mechanism of claim 1, comprising two or more arms.

4. The detachment mechanism of claim 1, wherein the arm is curved.

5. The detachment mechanism of claim 1, wherein the distal end of the arm further comprises a ball-like structure.

6. The detachment mechanism of claim 3, wherein in the first position, the arms are separated and engage the vaso-occlusive device.

7. The detachment mechanism of claim 1, further comprising a mandrel, which mandrel engages the arm with the vaso-occlusive device.

8. A detachment mechanism adapted to detachably engage a vaso-occlusive device, the detachment mechanism comprising
at least one arm having first and second positions, wherein in the first position, the arm engages the vaso-occlusive device and, in the second position, the arm releases the vaso-occlusive device, and
means for moving the detachment mechanism between the first and second positions.

9. A vaso-occlusive assembly comprising
a vaso-occlusive device having proximal and distal ends;
a pusher element having proximal and distal ends;
a detachment mechanism according to claim 8 at the distal end of the pusher element, wherein the arm of the detachment mechanism has a distal end and proximal end.

10. The vaso-occlusive assembly of claim 9, wherein the distal end of the arm engages the vaso-occlusive device and the proximal end of the arm is embedded in the pusher element.

11. The vaso-occlusive assembly of claim 9, wherein the vaso-occlusive device comprises a helically wound vaso-occlusive coil.

12. The vaso-occlusive assembly of claim 11, wherein the arm engages one or more proximal windings of the helically wound coil.

13. The vaso-occlusive assembly of claim 11, wherein the pitch of one or more proximal windings of the coil is modified to engage the arm.

14. The vaso-occlusive assembly of claim 9, wherein the vaso-occlusive device engages an anchor structure at the proximal end of the vaso-occlusive device, which anchor structure extends into the vaso-occlusive device.

15. The vaso-occlusive assembly of claim 14, wherein the anchor structure comprises a ring or loop.

16. The vaso-occlusive assembly of claim 9, further comprising a removeable mandrel holding the arm in the first, engaged position.

17. A method of at least partially occluding an aneurysm, the method comprising the steps of
introducing a vaso-occlusive assembly according to claim 9 into the aneurysm, wherein the detachment mechanism is in the engaged position; and
switching the detaching mechanism to the unengaged position, thereby instantaneously deploying the vaso-occlusive device into the aneurysm.
